(19) United States
(12) Patent Application Publication
(10) Pub. No.: US 2014/0024090 A1
(43) Pub. Date: Jan. 23, 2014

(54) METHOD AND APPARATUS FOR CONVERTING A CARBON SOURCE INTO A HYDROCARBON OR HYDROCARBON PRECURSOR

(71) Applicant: SHELL OIL COMPANY, Houston, TX (US)

(72) Inventors: Steven Daniel DOIG, Cornwall (GB); Steven Richard BROWN, Chester (GB); George Robert LEE, Chester (GB); David Alexander PARKER, Chester (GB)

(73) Assignee: SHELL OIL COMPANY, Houston, TX (US)

(21) Appl. No.: 13/715,502

(22) Filed: Dec. 14, 2012

(30) Foreign Application Priority Data
Dec. 19, 2011 (EP) EP11194333.8

(51) Int. Cl. C12P 7/00; C12M 1/00; C12P 5/00

(52) U.S. Cl. CPC C12P 7/00; C12P 5/00; C12M 21/00
USPC 435/135; 435/136; 435/148; 435/147; 435/155; 435/166; 435/303.1; 29/428

(57) ABSTRACT

Method for converting a carbon source such as a plant-derived material into a hydrocarbon or hydrocarbon precursor, via a microorganism-catalysed aerobic reaction. The microorganism is cultivated in the presence of the carbon source and of oxygen, in a reaction vessel having a capacity of about 2000 litres or greater which comprises an aeration system for supplying oxygen to the microorganism. Also provided is an apparatus for use in the method, comprising a reaction vessel of capacity 2000 litres or greater, and an aeration system.

### CROSS REFERENCE TO RELATED APPLICATIONS

This application claims the benefit of priority of European Patent Application No. 11194333.8, filed on Dec. 19, 2011, the disclosure of which is incorporated by reference herein in its entirety.

### FIELD OF THE INVENTION

Embodiments of this invention generally relate to a method and apparatus for converting a carbon source, such as a sugar, into a hydrocarbon or hydrocarbon precursor, and more particularly to such conversion using an aerobic microorganism.

### BACKGROUND TO THE INVENTION

This section is intended to introduce various aspects of the art, which may be associated with exemplary embodiments of the present invention. This discussion is believed to assist in providing a framework to facilitate a better understanding of particular aspects of the present invention. Accordingly, it should be understood that this section should be read in this light, and not necessarily as admissions of any prior art.

Hydrocarbons which may be of use as fuels can be produced via a microbial pathway, using organic feedstock such as plant-derived sugars. For example, microorganisms can be used to catalyse the fermentation of sugars derived from plant products such as corn and sugar cane, and the fermentation products (for instance oxygenates such as fatty acids, aldehydes and alcohols) can subsequently be converted to alkanes by chemical processes such as decarboxylation, reduction and hydrogenation. In certain situations, microorganisms have been found capable of catalysing hydrocarbon syntheses by purely biochemical routes: see for example “Aliphatic and isoprenoid hydrocarbon biosynthesis for diesel fuels”, Andrew C Murley, Department of Biochemistry and Molecular Biology, Michigan State University Basic Biotech, 2009 5:1, ISSN 1944-3277.

It is also known to cultivate microorganisms such as microalgae and certain types of yeast, in order to harvest the oils which they naturally secrete, or in order to extract oils from their cells. These microbial oils can then be hydrotreated to convert them into hydrocarbons, and used in the preparation of fuel components. Again, such microorganisms can be fed on carbon growth sources derived from biological materials, in particular plant products.

Fuels which can be produced from purely natural materials, for example from plant products or by microalgae, can be referred to as biofuels. They represent an attractive alternative to conventional mineral fuels, as they typically have a good energy balance (the amount of energy generated by combustion of a fuel minus the amount required to produce it). The demand for such fuels is therefore increasing rapidly, both in the interests of the environment and to comply with increasingly stringent regulatory demands and consumer expectations.

Many of the microorganisms which are capable of producing hydrocarbons—or hydrocarbon precursors such as microbial oils—from carbon sources, or at least those which do so efficiently, respire aerobically. They therefore need to be cultivated in a suitably equipped bioreactor, which can be supplied with oxygen. If they are to be used to generate fuel components on a commercial scale, then the bioreactor will need a large capacity, for example up to 5 million litres. At present, however, bioreactors are typically used on a much smaller scale. Increasing their capacity can bring complications due to the need to aerate large volumes of cultivation mixture. Inevitably, the efficient aeration of a large capacity vessel is likely to consume a large amount of energy. The air supply also needs to be well distributed throughout the cultivation mixture, which can be difficult to achieve in a large volume, particularly since microorganisms can be damaged by the use of high shear agitation systems.

Since the technology required to generate hydrocarbon fuels and their precursors from biological material is still in its infancy, solutions to such problems have as yet been little investigated. Processes have typically been carried out on a laboratory or at best pilot scale.

Larger scale bioreactors are known for use in the food industry, in particular for the production of ethanol by fermentation (for example for the brewing of beer). However, such fermentors have no need for an air supply as the fermentation process is carried out anaerobically. They would thus be unsuitable for the production of hydrocarbons using aerobic pathways.

In US-A-2010/0196994, there is described a method and apparatus for processing stillage derived from an ethanol-producing fermentation process. The system involves the use of a continuous flow airlift bioreactor, in which the stillage is treated with aerobic fungi in order to generate a clean, recyclable stream of water and a proteinaceous fungal biomass which can be incorporated into animal feed supplements. Pilot-scale reactors are described, with capacities of up to about 1300 litres, in which aeration is via a diffuser at the bottom of the reactor. The document also refers to the production of oils from the stillage using oleaginous moulds, again involving the use of an airlift bioreactor (fermentor), and to the use of such oils in the production of biofuels.

It would be an advancement in the art to provide a system for converting carbon sources into hydrocarbons and/or hydrocarbon precursors on a large, suitably commercial, scale, which system can overcome or at least mitigate the above described problems.

### SUMMARY OF THE INVENTION

According to a first aspect of the present invention there is provided a method for converting a carbon source into a hydrocarbon or hydrocarbon precursor via a microorganism-catalysed aerobic reaction, the method comprising cultivating the microorganism in the presence of the carbon source and of oxygen, wherein the cultivation is carried out in a reaction vessel of capacity 2000 litres or greater and the reaction vessel comprises an aeration system for supplying oxygen to the microorganism during its cultivation.

According to a second aspect of the invention, there is provided an apparatus for use in a method for converting a carbon source into a hydrocarbon or hydrocarbon precursor via a microorganism-catalysed aerobic reaction, the apparatus comprising (i) a reaction vessel of capacity 2000 litres or greater, and (ii) an aeration system for supplying oxygen to the vessel. Such apparatus may in particular be for use in a method according to the first aspect of the invention.

A third aspect of the invention provides a method for constructing apparatus which is for use in a method for converting a carbon source into a hydrocarbon or hydrocarbon precursor via a microorganism-catalysed aerobic reaction, the method comprising fitting a reaction vessel of capacity 2000 litres or greater with an aeration system for supplying oxygen to the vessel. The reaction vessel may previously have been used for, or suitable or adapted to be used for, a microorganism-catalysed fermentation reaction, such as for the production of ethanol. The resultant apparatus may be apparatus according to the second aspect of the invention.

A fourth aspect of the invention provides a process for producing a biofuel component, which process comprises the use of a conversion method according to the first aspect and/or apparatus according to the second aspect, in order to generate a hydrocarbon or hydrocarbon precursor which is suitable for use as, or conversion to, a biofuel component. The hydrocarbon or precursor may for instance be suitable for use as, or conversion to, a gasoline, kerosene or diesel fuel component.

In a method for converting a carbon source into a hydrocarbon or hydrocarbon precursor according to aspects of the present invention, the method comprises the step of cultivating a microorganism in the presence of a carbon source and oxygen to convert at least a portion of the carbon source into a hydrocarbon or hydrocarbon precursor. The cultivation is carried out in a reaction vessel having a capacity of about 2000 litres or greater and the reaction vessel comprises an aeration system for supplying oxygen to the microorganism during its cultivation.

In one embodiment, the microorganism is a micro-aerobic microorganism. In another embodiment, the microorganism is a high activity microorganism. In another embodiment, the carbon source comprises a plant-derived material. In another embodiment, the capacity of the reaction vessel is about 100,000 litres or greater. In yet another embodiment, the method further comprises the step of producing a biofuel component using the hydrocarbon or hydrocarbon precursor. In one embodiment, the method further comprises subjecting the hydrocarbon or hydrocarbon precursor to a downstream hydrotreatment.

In an apparatus for converting a carbon source into a hydrocarbon or hydrocarbon precursor according to aspects of the present invention, the apparatus comprises (i) a reaction vessel having a capacity of about 2000 litres or greater, and (ii) an aeration system for supplying oxygen to the vessel.

In one embodiment, the aeration system comprises a circulation loop, the loop comprising a first conduit in liquid communication with the reaction vessel, the first conduit is adapted to allow liquid to exit the reaction vessel; a second conduit in fluid communication with the reaction vessel, the second conduit is adapted to return the exited liquid to the reaction vessel; and an oxygen entrainment section between the first and second conduits adapted to allow contact between the liquid from the first conduit and oxygen prior to the liquid returning to the reaction vessel.

In one embodiment, the oxygen is contained in an oxygen-containing fluid selected from oxygen, air and oxygen-enriched air. In one embodiment, the circulation loop comprises a liquid propelling means for driving the liquid in the reaction vessel to flow through the loop in a desired direction. In one embodiment, the liquid propelling means is a pump. In one embodiment, the location where liquid exits the reaction vessel into the first conduit is positioned higher than the location where liquid enters the reaction vessel from the second conduit. In another embodiment, at least a portion of the circulation loop is positioned lower than the reaction vessel.

In one embodiment, the circulation loop further comprises a section having an essentially U-shape. In one embodiment, the apparatus further comprises a stirring element configured to cause movement in the liquid in the reaction vessel. In another embodiment, the apparatus further comprises a temperature controlling element.

In one embodiment, the liquid returning to the vessel reaction from the second conduit has a higher oxygen concentration than the liquid exiting the reaction vessel to the first conduit. In one embodiment, the higher oxygen concentration is caused at least by the depth at which a portion of the circulation loop is positioned below the reaction vessel.

In a method for constructing an apparatus according to aspects of the present invention, the method comprises converting a carbon source into a hydrocarbon or hydrocarbon precursor via a microorganism-catalysed aerobic reaction, the method comprising fitting a reaction vessel having a capacity of about 2000 litres or greater with an aeration system for supplying oxygen to the vessel.

Other features of embodiments of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

Embodiments of the invention may be better understood by reference to the drawings in combination with the detailed description of specific embodiments presented herein.

FIG. 1 shows a schematic diagram of a first example of an apparatus according to certain aspects of the invention.

FIG. 2 shows a schematic diagram of a second example of an apparatus according to certain aspects of the invention

### DETAILED DESCRIPTION OF PREFERRED EMBODIMENTS OF THE INVENTION

Embodiments of this invention provide a method and apparatus for converting a carbon source, such as a sugar, into a hydrocarbon or hydrocarbon precursor, using an aerobic microorganism. The microorganism used in certain embodiments of the invention may for example be selected from algae (in particular microalgae, also known as microphytes), fungi (including yeasts), bacteria, and mixtures thereof. In one embodiment, the microorganism is selected from bacteria, fungi, and any combination thereof. In another embodiment, the microorganism is an oil- or hydrocarbon-producing alga, preferably an oil- or hydrocarbon-producing microalga. In another embodiment, the microorganism is a fungus, which may be an oil- or hydrocarbon-producing fungus. In a preferred embodiment, the microorganism is a yeast. In the present context, the term “microalgae” and related terms includes at least diatoms and cyanobacteria. The term “oil- or hydrocarbon-producing” may mean that the microorganism secretes one or more oils and/or hydrocarbons, and/or that it generates one or more oils and/or hydrocarbons within its cells.

In a method according to certain aspects of the invention, the microorganism used is preferably an aerobic organism, which is of the type which consumes oxygen in order to respire and reproduce. In one embodiment, the microorganixm is a micro-aerobic microorganism, which can survive and reproduce in an atmosphere having a lower oxygen concentration than ambient air. The micro-aerobic microorganism may, for example, be capable of surviving in an atmosphere having an oxygen concentration of about 0.1 mmol per litre or less. In one embodiment, the use of such micro-aerobic microorganisms can help reduce the problems associated with aerating a large capacity reaction vessel, since lower oxygen concentrations may be needed throughout the cultivation mixture it contains. This in turn can increase the efficiency of certain embodiments of the invented method. The micro-aerobic microorganism can also reduce the amount of heat generated by the microorganism through respiration, which means that less of the energy available from the carbon source is wasted in heat generation, and which can also make it easier to control the temperature within the reaction vessel.

Non-limiting examples of suitable micro-aerobic microorganisms include for example strains of Saccharomyces cerevisiae. Anaerobic Strains of S. cerevisiae are already used in anaerobic fermentation processes such as for the brewing of alcohol drinks or the baking of bread and other yeast-containing food products. They can also, however, in appropriate conditions (for example with limited access to glucose), be induced to respire aerobically, and thus to generate products other than ethanol: in this way they can be used, in the method of the present invention, to convert a suitable carbon source into a hydrocarbon or hydrocarbon precursor.

In another embodiment, the microorganism is preferably a high activity microorganism. In this context the term “high activity” can be defined as capable of an activity of about at least 0.01 mol carbon consumed per gram of biomass per hour in the aerobic reaction according to aspects of the invention.

Non-limiting examples of suitable high activity microorganisms include for example E. coli, Saccharomyces cerevisiae, Bacillus subtilis, Lactobacillus species, Lactococcus lactis, Actinomycetes species, Streptococcus species, Kluyveromyces lactis, Kluyveromyces marxianus, Pichia stipitis, Yarrowia lipolytica and Campylobacter species.

In one embodiment, the microorganism may be of the type that can convert biomass (ie plant-derived matter), or at least a carbon-containing component thereof such as a sugar, into one or more microbial oils and/or into one or more microbial hydrocarbons such as alkanes or alkenes. Such microorganisms include those listed above. It may be a microorganism which secretes an oil and/or a hydrocarbon. It may be a microorganism from which an oil and/or hydrocarbon can be extracted, for example by lysis of its cells. Microbial oils can be converted, preferably by hydrotreatment, into hydrocarbons such as alkanes, alkenes, cyclic hydrocarbons, naphthenic hydrocarbons and aromatic hydrocarbons, and thus rendered suitable for use as fuel components.

In a method according to aspects of the invention, the carbon source may be any carbon-containing component, or any combination thereof, which the microorganism is capable of converting into a hydrocarbon or hydrocarbon precursor via an aerobic reaction pathway. The carbon source may in particular comprise a plant-derived material. It may for example be selected from sugars (for example glucose, maltose, fructose, pentoses and hexoses), alcohols (in particular glycerol and other polyols), carboxylic acids (in particular fatty acids), carbonates, and any combination thereof. In one embodiment, the carbon source comprises one or more sugars. In another embodiment, it comprises glucose.

A method according to aspects of the invention may comprise cultivating the microorganism in the presence of biomass from a suitable source, including for example a processed source, such as paper. The biomass may comprise, or derive from, a material selected from straw (for example wheat straw or rice straw); corn and corn products such as corn stover, corn fibre and corn cobs); bagasse; sugar cane; wood and wood residues; nut shells; grasses such as switchgrass and miscanthus; paper; cotton seed hairs; plant material from sorted refuse; and any combination thereof. In an embodiment, the biomass comprises, or derives from, a material selected from straw, corn products, bagasse, wood residues, and any combination thereof.

Prior to carrying out the method of the invention, such biomass may have been treated, for example, to reduce the size of particles or other solid elements contained within it, and/or to increase its homogeneity. The biomass may, for example, have been shredded, milled, ground and/or compacted. Instead or in addition, the biomass may have been chemically or biochemically treated in order to render one or more of its carbon-containing components more readily available for conversion by the microorganism: it may for example have been subjected to a hydrolysis process, in particular an enzymatic hydrolysis, in order to convert one or more of its components (preferably a cellulose) into a sugar.

During embodiments of the invented method, the microorganism may suitably be present in the reaction vessel as a constituent of a cultivation mixture containing the carbon source, oxygen and preferably also water, optionally with one or more further nutrients and/or processing additives such as antifoaming additives. The cultivation mixture may for instance take the form of a fermentation broth, where the microorganism is used to ferment the carbon source or a material containing it. The cultivation mixture may also contain one or more microbial products, which are generated by the microorganism as it respires and reproduces, in particular one or more microbial oils and/or hydrocarbons.

A method according to aspects of the invention may suitably comprise loading the reaction vessel with an appropriate quantity of the microorganism, which may have been grown from an inoculum in known fashion or may have been procured in a form ready for use in the method. The method may also suitably comprise loading the reaction vessel with an appropriate quantity of the carbon source, for example with an appropriate quantity of biomass. During cultivation of the microorganism, the vessel may be supplied with oxygen—or at least with a fluid (for example air) which contains oxygen—via an aeration system. It may also be supplied with one or more nutrients suitable for sustaining the microorganism. In one embodiment, such nutrients comprise the carbon source which is to be converted into the hydrocarbon or hydrocarbon precursor. For example, before or during cultivation of the microorganism the reaction vessel may be supplied with an optionally pretreated biomass feed, and/or with a sugar feed. One or more other nutrients may be supplied, as are suitable for the chosen microorganism: such nutrients may for example be selected from sugars, salts, vitamins, minerals, and mixtures thereof.

The supply of oxygen, carbon source and/or other nutrients (including of biomass where applicable) during cultivation of the microorganism may be done on a continuous basis or batch-wise. Thus, for example, the reaction vessel may be supplied continuously with oxygen or an oxygen-containing fluid. It may be supplied continuously with a nutrient feed, or alternatively a discrete quantity of the carbon source and/or other nutrient(s) may be added to the vessel more than once, at intervals during the cultivation.

The cultivation may suitably be carried out in an aqueous medium; the invented method may therefore comprise loading the reaction vessel with water, or with an aqueous solution for example of nutrients, as well as with the microorganism. The cultivation mixture may suitably be maintained at a temperature, pressure and pH which are suitable to allow, and ideally to promote, microorganism reproduction. The microorganism may suitably be cultivated in the dark. It may suitably be cultivated at a temperature from about 5 to 85° C., and/or at a pressure of from about 1 to 2 atmospheres (about 0.1 to 0.2 MegaPascal), more preferably at about 1 atmospheric pressure (about 0.1 MegaPascal).

The method may comprise removing one or more components from the reaction vessel during the cultivation, again either continuously or at intervals. It may comprise removing one or more waste products such as carbon dioxide and/or oxygen-depleted air, for example by venting to atmosphere. It may comprise harvesting one or more products such as hydrocarbons or hydrocarbon precursors from the reaction vessel, which may suitably be achieved by removing a quantity of the cultivation mixture from which to extract the relevant product(s).

Therefore, one embodiment of the invention comprises removing one or more portions of the cultivation mixture from the reaction vessel, either continuously or at intervals. The removed portions may contain microorganism and relevant hydrocarbon(s) or hydrocarbon precursor(s), and preferably one or more other components such as water, nutrients, carbon source (for example biomass) and/or optional processing additives. From the removed portions, desired products can be separated using known separation techniques. If appropriate, other components such as live microorganism, nutrients or water can be separated from the removed portions and recycled back into the reaction vessel. Components of the removed portions may be subjected to one or more downstream processes such as purification, cleaning or further chemical or biochemical reactions. In particular, a hydrocarbon precursor such as a microbial oil may be separated from a removed portion, and then hydrotreated to convert it into a hydrocarbon.

Hydrocarbons and their precursors can be separated from removed portions using any of a range of suitable techniques, for example selected from centrifugation, solvent extraction, distillation, membrane extraction, hydrocyclone extraction, and combinations thereof. A separated hydrocarbon or hydrocarbon precursor may then be subjected to further downstream processing such as hydrotreatment.

In one embodiment, following removal of a portion of the cultivation mixture, the microorganism within it may be subjected to a lysis procedure, in order to release a microbial product such as an oil or hydrocarbon from within its cells. Lysis may be achieved using any suitable method, for example using an enzymatic, physical, chemical, osmotic or mechanical method or combination thereof. The microbial product may then be separated from the resultant lysate mixture, as described above.

In another embodiment, a removed portion of cultivation mixture may be subjected to a concentration step, suitably before it is subjected to lysis and/or before a hydrocarbon or hydrocarbon precursor is separated from it. It may for instance be concentrated using a technique selected from centrifugation, filtration, flotation, coalescence, sedimentation, flocculation, decantation, cyclone separation, and combinations thereof.

In the context of the present invention, a hydrocarbon is an organic component containing only carbon and hydrogen atoms. A hydrocarbon may for instance be selected from alkanes, alkenes, cyclic hydrocarbons, naphthenic hydrocarbons, aromatic hydrocarbons, and mixtures thereof, in particular from alkanes, alkenes, and mixtures thereof. A “hydrocarbon precursor” is an organic component which can subsequently be converted into a hydrocarbon, for example by hydrotreatment.

A hydrocarbon precursor may comprise a microbial oil. Such an oil may be suitable for use as, and/or conversion to, a fuel. It may be suitable for use as, or conversion to, a biodiesel, biokerosene or biogasoline fuel, or a biocomponent of a diesel, kerosene or gasoline fuel formulation.

A hydrocarbon precursor may comprise an oxygenate, for example selected from alcohols (including fatty alcohols); fatty acids; mono-, di- and triglycerides; lipids; ketones; aldehydes; and any combination thereof. It may in particular be selected from fatty acids, alcohols, and any combination thereof.

A hydrocarbon precursor may be convertable into a hydrocarbon using a process selected from hydrogenation, (hydro)isomerisation, dehydration, hydro-deoxygenation, cyclisation, catalytic cracking, and any combination thereof. To render such a precursor suitable for use as or in a fuel, it may in addition be subjected to a process selected from esterification, etherification, desulphurisation, denitrification, and any combination thereof.

Embodiments of the invention may be used to convert one or more carbon sources into one or more hydrocarbons or hydrocarbon precursors. They may therefore be used to produce a mixture of hydrocarbons and/or hydrocarbon precursors.

In one particular embodiment, the carbon source is converted into one or more hydrocarbons which are suitable for use in or as fuel components, for example in or as gasoline, kerosene or diesel fuel components. In another embodiment, it is converted into one or more hydrocarbon precursors (in particular microbial oils) which can be hydrotreated to form hydrocarbons which are suitable for use in or as fuel components. Hydrocarbons which are suitable for use in or as gasoline, kerosene or diesel fuel components may for example contain from 4 to 19 carbon atoms.

In the method of the invention, the reaction vessel may for example have a capacity of about 5000 litres or greater, or of about 10000 or 50000 or 100000 litres or greater. It may for example have a capacity of up to about 1.5 million litres, or of up to about 1.3 million litres, or of up to about 1 million or 750000 or 500000 litres, such as from about 2000 to 1.5 million litres or from about 5000 to 500000 litres.

Embodiments of the present invention include an aeration system, by which the microorganism in the reaction vessel is supplied with oxygen. The aeration system may be of any suitable type. In one embodiment, it supplies the reaction vessel with atmospheric air, for example with air from the environment which surrounds the vessel. The rate at which the oxygen is supplied to the vessel may be chosen based on factors such as the nature of the microorganism, the capacity of the vessel and the amount of material inside it. The oxygen may for example be supplied at a rate of about at least 0.5 mmol per litre of cultivation mixture within the vessel per hour, or of about 1 mmol/litre/hour or more. It may for example be supplied at a rate of up to about 10 mmol/litre/hour, or of up to about 7.5 or 5 mmol/litre/hour, such as from about 1 to 10 mmol/litre/hour or about 10 mmol/litre/hour. The oxygen may be supplied at a rate which increases during cultivation of the microorganism, to cater for the increasing number of oxygen-consuming microorganism cells.

In one embodiment, the aeration system supplies oxygen to the lower part of the reaction vessel, for instance via its base.

In another embodiment, the aeration system comprises a pump for pumping oxygen, air or another oxygen-containing fluid from outside the reaction vessel into the cultivation mixture within it. The outlet from such a pump may be conventional in construction and may for instance comprise a plurality of apertures through which the oxygen-containing fluid may be delivered to different locations within the vessel, so as to assist in achieving homogeneous aeration of the cultivation mixture.

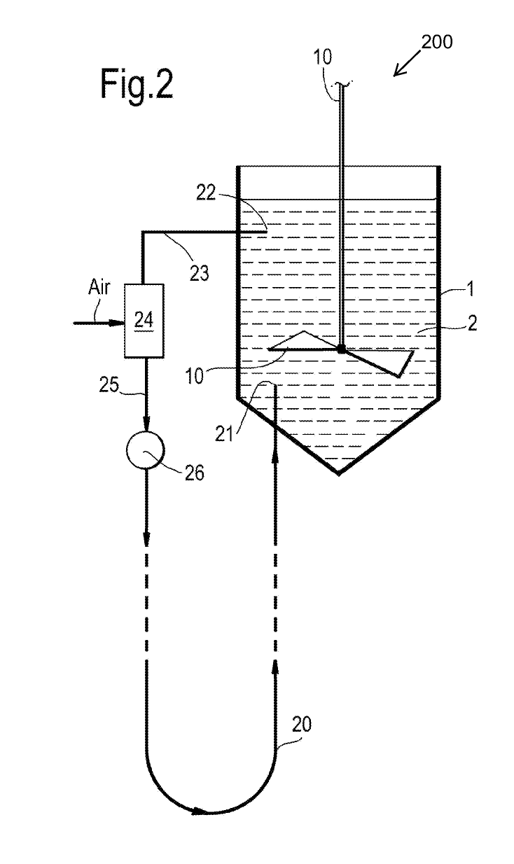

In one embodiment, the aeration system comprises a liquid circulation loop with an oxygen entrainment section, such as of the type described below in connection with FIG. 2.

A method according to aspects of the invention may suitably comprise controlling the temperature within the reaction vessel, to maintain it within a desired operating range. The cultivation of the microorganism may for example be an exothermic process, and the method may then involve cooling the reaction vessel in order to maintain the cultivation mixture within a desired temperature range which is suitable for microorganism reproduction. Conventional means may be used to achieve such temperature control, for example selected from heaters, heat exchangers, heating and/or cooling jackets, and combinations thereof. Heat which is extracted from the vessel may be used in another processing step, for example in the distillation of a hydrocarbon or hydrocarbon precursor product of the method.

In one embodiment, the method may comprise controlling the pressure within the reaction vessel, to maintain it within a desired operating range. Conventional means may be used to achieve such pressure control, for example selected from pressure regulators, flow control valves, and combinations thereof.

In another embodiment, the method may suitably comprise stirring the cultivation mixture, to increase its homogeneity in particular in terms of microorganism access to oxygen, carbon source and/or other nutrients. Conventional means such as paddles and baffles may be used to achieve the desired degree of movement within the cultivation mixture. Suitably, however, the nature and degree of such stirring may generate sufficiently low levels of shear energy, in the cultivation mixture, as to avoid damage to the microorganism.

In one embodiment, the method of the invention may be carried out as a batch process, or more particularly as a semi-continuous process. In the present context, a semi-continuous process is one in which the microorganism cultivation is allowed to continue in the reaction vessel for a period, the desired hydrocarbon or hydrocarbon precursor product being removed during the cultivation period either continuously or at intervals but without emptying the vessel of the microorganism during that period. As described above, such a process may involve continuing to supply the microorganism with oxygen, and optionally with nutrients, during the cultivation period. It may involve removing and optionally recycling components such as microorganism or water during the cultivation period. The microorganisms which are present during such a semi-continuous process may all be derived, directly or indirectly, from an inoculum introduced into the reaction vessel at the start of the invented method.

According to a second aspect of the invention, there is provided apparatus for use in a method for converting a carbon source into a hydrocarbon or hydrocarbon precursor via a microorganism-catalysed aerobic reaction, the apparatus comprising (i) a reaction vessel with a capacity of about 2000 litres or greater, and (ii) an aeration system for supplying oxygen to the vessel. Such apparatus may in particular be for use in a method according to the first aspect of the invention.

In one embodiment, the aeration system may, as described in connection with the first aspect of the invention, supply air to the reaction vessel, suitably atmospheric air. It may comprise a pump. It is suitably capable of delivering oxygen to the reaction vessel at a rate of from about 1 to 10 mmol per litre per hour.

In one embodiment, the aeration system comprises an oxygen-entraining liquid circulation loop. Such a loop comprises a first conduit through which liquid (preferably cultivation mixture) can exit the reaction vessel; a second conduit through which liquid which has exited the vessel may return to it; and an oxygen entrainment section between the first and second conduits, in which the liquid can be brought into contact with an oxygen-containing fluid (in particular a gas) so as to allow oxygen to enter the liquid prior to the liquid returning to the reaction vessel. The fluid may for example be selected from oxygen, air and oxygen-enriched air. The second conduit may simply be a continuation of, or otherwise in fluid communication with, the first conduit.

Such an aeration system can be used to supply a relatively low but substantially constant level of oxygen to the reaction vessel, with relatively little energy input compared to many known aeration systems.

The liquid circulation loop may comprise a liquid propelling means by which liquid can be driven to flow through the loop, out of and back into the vessel, in a desired direction. The liquid propelling means may comprise a pump.

Suitably, the first conduit may allow liquid to exit the reaction vessel at a location which is vertically spaced from the location at which the second conduit reintroduces liquid into the vessel. For example, the first conduit may allow liquid to exit the reaction vessel at a location which is vertically above the location at which the second conduit reintroduces the liquid. In this way, gravity can help to keep liquid circulating between the loop and the vessel, and oxygen can be drawn into the flowing liquid as it passes through the oxygen entrainment section. Suitably the inlet to the first conduit and the outlet from the second conduit are spaced a relatively small vertical distance apart, so as to reduce the energy required to drive liquid flow through the loop.

In an embodiment where the reaction vessel comprises stirring means (for example one or more impellers) for agitating the cultivation mixture within it, suitably the first conduit may allow liquid to exit the reaction vessel at a location which is vertically above the stirring means. In such a situation, the second conduit may suitably reintroduce liquid into the vessel at a location which is vertically below the stirring means. A stirring means such as an impeller might suitably be spaced, in use, from the bottom of the reaction vessel by a vertical distance approximately equal to one sixth of the depth of the cultivation mixture within the vessel, or by a vertical distance approximately equal to one third of the impeller diameter.

Suitably, the liquid circulation loop may be arranged such that liquid can flow vertically downwardly through and/or downstream of the oxygen entrainment section. The term “vertically downwardly” in this context means that at least a component of the velocity of the flowing liquid is in a vertical direction.

The oxygen entrainment section is suitably positioned at a location which is vertically below the location at which liquid exits the reaction vessel through the first conduit, but vertically above the location at which liquid re-enters the vessel through the second conduit.

In one embodiment, a lower portion of the liquid circulation loop is positioned vertically below the base of the reaction vessel. It may for example be positioned about 1 m or more below the base of the reaction vessel, or about 5 or 10 m or more, or about 25 or 50 or 75 m or more. It may for example be positioned up to about 150 m below the base of the reaction vessel, or up to about 125 or 100 m below, or in cases up to about 75 or 50 m below. In an embodiment, at least a portion of the loop is positioned from about 5 to 100 m below the base of the reaction vessel, or from about 10 to 100 m below, or from about 5 to 50 m below. In this way, entrained oxygen can be dissolved in the flowing liquid to a relatively high concentration, due to the high hydrostatic pressure in the lower portion of the loop. This can allow more efficient delivery of oxygen to the reaction vessel without the need for high energy pumps.

Some or all of those parts of the loop which are located vertically below the reaction vessel base may be buried in the ground, for example below, or adjacent to, the vessel.

In a particular embodiment, the liquid circulation loop comprises a substantially U-shaped tube, the lowest point of which is positioned below the base of the reaction vessel as described above. The two arms of the U-shaped tube may extend vertically upwards: one may for example connect to, or form part of, the first conduit by which liquid can exit the reaction vessel, and one may for example connect to, or form part of, the second conduit by which liquid can be reintroduced into the vessel. The oxygen entrainment section may then suitably be positioned in the arm which connects to or forms part of the first conduit, downstream of the reaction vessel in the direction of liquid flow.

Such a U-shaped tube may be located at least partly within the reaction vessel, for instance with the open end of one of its arms positioned at the base of the vessel and thus, in use, within a cultivation mixture contained in the vessel. Alternatively, the U-shaped tube may be located remotely from the reaction vessel, and connected thereto by means of suitable conduits.

The oxygen entrainment section of the aeration system may comprise a fluid (in particular gas) inlet in a conduit which forms part of the liquid circulation loop. It may comprise a fluid inlet in a chamber through which liquid passes as it flows around the loop, such that the liquid can come into contact with fluid which enters the chamber through the fluid inlet. It may comprise a pumped or otherwise forced fluid inlet system through which a fluid (in particular a gas) may be introduced into liquid passing through the oxygen entrainment section. In an embodiment the oxygen entrainment section may comprise a turbine unit.

A conduit which forms part of the liquid circulation loop, in particular of a U-shaped tube as described above, may suitably have an internal cross section of an area which is significantly smaller than that of the reaction vessel. An appropriate area may be chosen to suit the capacity and operating rate of the overall system, and the hydrostatic pressure which it is desired to generate within the relevant conduit. Such a conduit may have a circular or approximately circular internal cross section.

In apparatus according to the second aspect of the invention, the aeration system may comprise more than one liquid circulation loop. Such loops may for instance communicate with the inside of the reaction vessel at different locations, so as to increase the homogeneity of the aeration.

An apparatus according to aspects of the invention may additionally comprise one or more of the following:

iii) nutrient supply means or element, by which one or more nutrients (which may comprise the carbon source which is to be converted into a hydrocarbon or hydrocarbon precursor) can be supplied to the vessel;

iv) stirring means or element, for causing movement within a microorganism-containing cultivation mixture within the reaction vessel;

v) temperature control means or element, for controlling the temperature within the reaction vessel;

vi) pressure control means or element, for controlling the pressure within the reaction vessel;

vii) a vent, through which a gas such as carbon dioxide can be released from the vessel;

viii) component removal element, by which one or more components can be removed from the reaction vessel; and/or

iv) component recirculation element, by which one or more components (for example cultivation mixture) can be returned to the reaction vessel after removal therefrom.

In another embodiment, the apparatus may also comprise one or more sensors and associated control means, by which conditions within the reaction vessel (for example oxygen concentration and/or temperature) may be monitored and controlled.

The nutrient supply means may comprise a source of a suitable nutrient or nutrient mixture, together with a conduit for supplying the nutrient(s) from the source to the reaction vessel. It may comprise a pump and/or flow control valve.

The stirring means may be as described above in connection with the first aspect of the invention: it may for example comprise one or more paddles or other rotatable stirrers, and/or baffles or other internal surface perturbations within the vessel.

The temperature and pressure control means may be as described above in connection with the first aspect of the invention. The temperature control means may for example comprise a heater, heat exchanger, heating or cooling jacket, or combination thereof, suitably in combination with a temperature sensor. The pressure control means may comprise a regulator, flow control valve or combination thereof, suitably in combination with a pressure sensor. Elements of the temperature and/or pressure control means may be positioned either inside or outside the reaction vessel, as appropriate.

The vent may suitably be located at or near the top of the reaction vessel.

A component removal means may for example comprise a conduit through which material(s) may be removed from the reaction vessel. It may comprise means for separating and/or purifying one or more components from a mixture of materials removed from the vessel.

A component removal means may suitably communicate with the inside of the reaction vessel at a location at or near the bottom of the vessel, for example so that in use it is below the surface of a cultivation mixture contained within the vessel.

A component recirculation means may comprise means for separating, cleaning and/or purifying a component (for example a microorganism or water) from a mixture of components removed from the reaction vessel. It may comprise a conduit by which such a component can be returned to the reaction vessel.

A component removal or recirculation means may comprise one or more pumps or flow control valves for controlling the flow of materials through it.

Apparatus according to the second aspect of the invention may be constructed by retrofitting an existing large scale reaction vessel, such as a fermentor, with an aeration system. In particular a fermentor may be fitted with an aeration system comprising a liquid circulation loop of the type described above.

Accordingly, a third aspect of the invention provides a method for constructing apparatus which is for use in a method for converting a carbon source into a hydrocarbon or hydrocarbon precursor via a microorganism-catalysed aerobic reaction, the method comprising fitting a reaction vessel having a capacity of about 2000 litres or greater with an aeration system for supplying oxygen to the vessel. The reaction vessel may previously have been used for, or suitable or adapted to be used for, a microorganism-catalysed fermentation reaction, such as for the production of ethanol. The resultant apparatus may be apparatus according to the second aspect of the invention.

A method or apparatus according to aspects of the present invention may be used to produce a hydrocarbon or hydrocarbon precursor which is suitable for use as, or conversion to, a biofuel component. Embodiments of the present invention may be used to produce a hydrocarbon precursor which can be converted (for instance by hydrotreatment) to a hydrocarbon which is then suitable for use as, or conversion to, a biofuel component. Thus a fourth aspect of the invention provides a process for producing a biofuel component, which process comprises the use of a conversion method and/or apparatus according to aspects of the present invention, in order to generate a hydrocarbon or hydrocarbon precursor which is suitable for use as, or conversion to, a biofuel component. The hydrocarbon or precursor may for instance be suitable for use as, or conversion to, a gasoline, kerosene or diesel fuel component.

A process according to the fourth aspect of the invention may comprise one or more downstream treatment steps by which the hydrocarbon, or in particular the hydrocarbon precursor, is rendered more suitable for use as a biofuel and/or otherwise modified and/or combined with one or more additional fuel components. Such treatment steps may for example be selected from hydrogenation, (hydro)isomerisation, dehydration, hydro-deoxygenation, cyclisation, catalytic cracking, esterification, etherification, desulphurisation, denitrification, and any combination thereof.

As in other embodiments of the invention, the microorganism used in the carbon source conversion may in particular be any suitable microorganism, such as a micro-aerobic and/or high activity microorganism.

Throughout the description and claims of this specification, the words “comprise” and “contain” and variations of the words, for example “comprising” and “comprises”, mean “including but not limited to”, and do not exclude other moieties, additives, components, integers or steps. Moreover the singular encompasses the plural unless the context otherwise requires: in particular, where the indefinite article is used, the specification is to be understood as contemplating plurality as well as singularity, unless the context requires otherwise.

Preferred features of each aspect of the invention may be as described in connection with any of the other aspects. Other features of the invention may become apparent from the following examples. Generally speaking the invention extends to any novel one, or any novel combination, of the features disclosed in this specification (including any accompanying claims and drawings). Thus features, integers, characteristics, compounds, chemical moieties or groups described in conjunction with a particular aspect, embodiment or example of the invention are to be understood to be applicable to any other aspect, embodiment or example described herein unless incompatible therewith. Moreover unless stated otherwise, any feature disclosed herein may be replaced by an alternative feature serving the same or a similar purpose.

Where upper and lower limits are quoted for a property, for example for the concentration of a fuel component, then a range of values defined by a combination of any of the upper limits with any of the lower limits may also be implied.

In this specification, references to properties such as solubilities, liquid phases and the like are—unless stated otherwise—to properties measured under ambient conditions, ie at atmospheric pressure and at a temperature of from about 18 to 25° C., for example about 20° C.

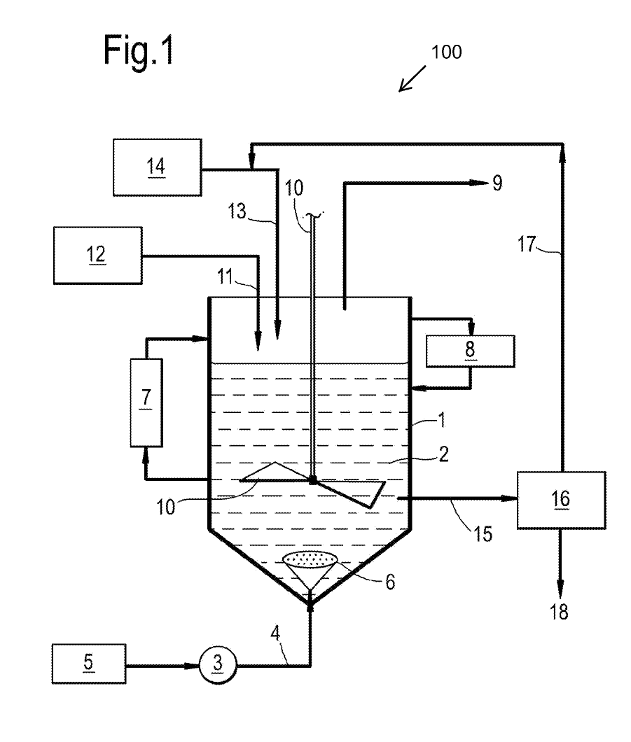

The present invention will now be further described with reference to the following non-limiting examples and the accompanying figures, of which FIGS. 1 and 2 show schematically two different sets of apparatus according to certain aspects of the invention.

### EXAMPLES

Referring FIG. 1, there is shown apparatus 100 according to aspects of the invention. Apparatus 100 includes reaction vessel 1, in which a microorganism can be cultivated in an aqueous medium (cultivation mixture) as indicated generally as 2. As shown, reaction vessel 1 is equipped with an aeration system that comprises pump 3 positioned in conduit 4 between air source 5 and perforated dispenser 6. Air can be pumped through the apertures in dispenser 6, into cultivation mixture 2.

Referring to FIG. 1, reaction vessel 1 is also equipped with means for controlling the temperature inside it. This is shown schematically as 7, in the form of a feedback loop which may comprise conventional equipment such as a heat exchanger, coupled to a temperature sensor inside the vessel (not shown) and suitable control means. Means for controlling the pressure inside reaction vessel 1 is shown schematically as 8, in the form of a feedback loop which may comprise conventional equipment such as a pressure regulator and associated control means. Outlet vent 9 allows gases such as carbon dioxide and oxygen-depleted air to be vented from reaction vessel 1, and this too can be used to assist in controlling the pressure, via appropriately positioned pressure gauges, pressure regulators, valves and the like (not shown).

In one embodiment, as shown in FIG. 1, stirrer 10 can be used to help increase the homogeneity of the cultivation mixture and the dispersion of oxygen and nutrients within it. Loading conduit 11, at the top of reaction vessel 1, may be used to load the vessel with a microorganism inoculum from source 12. A second loading conduit 13 may be used to load reaction vessel 1 with for instance nutrients, water and/or carbon-containing energy sources such as sugars or plant biomass, from sources indicated generally as 14.

Referring to FIG. 1, unloading conduit 15 may be used to remove a quantity of cultivation mixture 2, which may then be passed to a downstream processing region generally labelled as 16, at which one or more components of the mixture may be separated, purified and/or otherwise treated. In one embodiment, recycling conduit 17 may be used to return suitably purified microorganism and/or water to reaction vessel 1 via loading conduit 13. The desired hydrocarbon or hydrocarbon precursor product may be removed from the system at 18, and if desired subjected to further treatment steps and/or put into use as a biofuel component.

In one embodiment, all conduits can be provided with flow controls such as valves, and/or pumps, as desired: such components may be entirely conventional and so are omitted from FIG. 1 for clarity.

In one embodiment, reaction vessel 1 comprises an existing ethanol fermentor that has been fitted with pump 3, conduit 4, air source 5, and perforated dispenser 6. It may have a capacity of from about 2000 to 1.5 million litres, depending on its intended use.

FIG. 2 shows another embodiment, apparatus 200, in accordance with aspects of the invention. This is in many respects the same as that of FIG. 1, and for clarity the common parts (for example the loading and unloading conduits and the temperature and pressure controls) have been omitted from FIG. 2. A key difference between the two types of apparatus is the aeration system, which in the FIG. 2 apparatus comprises a liquid circulation loop with an air entrainment section.

Referring to FIG. 2, the circulation loop comprises tube section 20, which is preferably essentially U-shaped. Tube section 20 is preferably located beneath reaction vessel 1. In one embodiment, tube section 20 extends below the base of reaction vessel 1 by between about 1 and 75 m, suitably from about 5 to 50 m. As shown, tube 20 has outlet 21 disposed in reaction vessel 1, which in use is within liquid cultivation mixture 2. The loop also has inlet 22, which again is in cultivation mixture 2. Inlet 22 leads, via tube 23, to air entrainment section 24, at which atmospheric air can be drawn into liquid flowing through the loop. From air entrainment section 24, another tube, tube 25 (which may be a continuation of tube 23) leads to tube section 20. In one embodiment, as shown in FIG. 2, pump 26 drives the liquid circulation. Pump 26 needs only be sufficiently powerful to counter the relatively small vertical separation between inlet 22 and the outlet.

In use, pump 26 drives the liquid from cultivation mixture 2 to flow from inlet 22, through tubes 23 and 25, and tube section 20, and through outlet 21 back into reaction vessel 1, as shown by the arrows. As the particular portion of cultivation mixture 2 passes through air entrainment section 24, it incorporates air before flowing through tube section 20. In one embodiment, because of its depth below reaction vessel 1, the liquid in tube section 20 is under a relatively high hydrostatic pressure which dissolves the oxygen from the entrained air in the liquid to a relatively high concentration, potentially becoming completely dissolved. In one embodiment, tube 20 is preferably essentially U-shaped to allow liquid to travel to a particular depth below reaction vessel 1 and return back to reaction vessel 1. In one embodiment, the depth is preferably at least the depth that achieves a desired dissolved oxygen concentration. Liquid re-entering reaction vessel 1 at 21 is therefore oxygen-rich and helps contribute to aeration of the whole of the cultivation mixture.

Such a system as shown can provide an efficient way of aerating the contents of the reaction vessel, at least to the level required by a micro-aerobic or high activity microbial catalyst.

Tube section 20 may be buried in the ground beneath, or adjacent to, the reaction vessel. It is also possible, in apparatus according to the invention, for parts of the liquid circulation loop (in particular the lower tube sections and/or the oxygen entrainment section) to be located remotely from reaction vessel 1, so long as they remain in fluid communication with cultivation mixture 2 inside vessel 1.

In one embodiment, tubes or tube sections 20, 23 and 25 may for example comprise cylindrical tubes. They preferably have a smaller internal cross sectional area relative to that of reaction vessel 1. The difference in internal cross sectional area serves to increase the pressure under which oxygen is dissolved into the circulating liquid, and to ensure a reasonable fluid flow rate.

In one embodiment, apparatus 200 can be constructed by fitting the aeration system having elements 20-26 to an existing ethanol fermentor.

Apparatus 100 and apparatus 200 may be used to carry out a method according to aspects of the invention. Referring for example to apparatus 100 of FIG. 1, reaction vessel 1 may firstly be loaded with an inoculum of the desired microorganism (suitably a micro-aerobic microorganism, or a high activity aerobic microorganism, for example a fungus) from source 12, via loading conduit 11. The microorganism may preferably be loaded in an aqueous suspension, together with appropriate nutrients. Also loaded into vessel 4 at this stage, from source 14 and via conduit 13, may be a carbon source on which the microorganism can feed: this may comprise a sugar and/or a source of sugar such as plant biomass.

The microorganism is then maintained under conditions which allow, and ideally promote, its reproduction. As the cultivation proceeds, air is supplied to cultivation mixture 2 via elements 3, 4, 5, 6, of the aeration system whilst carbon dioxide and oxygen-depleted air are removed to vent 9. Further nutrients may be supplied to the microorganism via loading conduit 13 as and when necessary. In one embodiment, stirrer 10 is operated to ensure thorough mixing of the components in the vessel and homogeneous aeration of the mixture in reaction vessel 1.

Temperature and pressure controls 7 and 8 are used to ensure the inside of reaction vessel 1 is at the correct temperature and pressure both at the time when the microorganism is loaded and also while it is being cultivated.

If the method is being operated batch-wise, the microorganism is cultivated for a desired period of time, following which its cultivation is stopped and the contents of the vessel removed. The desired hydrocarbon or hydrocarbon precursor product may be separated from the removed components, using conventional separation techniques such as solvent extraction or distillation.

If the method is being operated semi-continuously, portions of cultivation mixture 2 may be removed via unloading conduit 15, either continuously as cultivation proceeds or at intervals during the cultivation period. From these removed portions the desired hydrocarbon or hydrocarbon precursor may be separated, again using conventional techniques. Other components, such as live microorganism, water and/or biomass, may be separated, cleaned and/or purified as appropriate, and if desired recycled back to reaction vessel 1 via recycling conduit 17.

It can be seen that embodiments of the present invention can make possible the biocatalysed production of hydrocarbon products, for example hydrocarbons which are of use in or as biofuels, on a commercial scale. Reaction vessels having capacities of several thousand litres can be used to carry out the cultivation process, provided they are equipped with suitable aeration systems. The use of a micro-aerobic and/or high activity microorganism can make it possible to aerate even large scale vessels to a sufficient extent for the necessary reactions to proceed. The use in particular of a liquid circulation loop of the type shown in FIG. 2 can provide a highly energy-efficient way of aerating the cultivation mixture.

Moreover, an existing fermentation vessel, used for example for the fermentation of plant-derived sugars to alcohols, can be modified with relative ease to allow its use in the aerobic hydrocarbon production method according to aspects of the invention. The modification may for instance involve connecting the vessel, via suitable fluid lines, to a circulation loop and air entrainment port, which may be located either adjacent to or remotely from the vessel.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as the presently preferred embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

What is claimed is:

1. A method for converting a carbon source into a hydrocarbon or hydrocarbon precursor, the method comprising the steps of:
cultivating a microorganism in the presence of a carbon source and oxygen to convert at least a portion of the carbon source into a hydrocarbon or hydrocarbon precursor, wherein the cultivation is carried out in a reaction vessel having a capacity of about 2000 litres or greater and the reaction vessel comprises an aeration system for supplying oxygen to the microorganism during its cultivation.

2. The method of claim 1, wherein the microorganism is a micro-aerobic microorganism.

3. The method of claim 1, wherein the microorganism is a high activity microorganism.

4. The method of claim 1, wherein the carbon source comprises a plant-derived material.

5. The method of claim 1, wherein the capacity of the reaction vessel is about 100,000 litres or greater.

6. An apparatus for converting a carbon source into a hydrocarbon or hydrocarbon precursor, the apparatus comprising:
(i) a reaction vessel having a capacity of about 2000 litres or greater, and
(ii) an aeration system for supplying oxygen to the vessel.

7. The apparatus of claim 6, wherein the aeration system comprises a circulation loop, the loop comprising:
a first conduit in liquid communication with the reaction vessel, the first conduit is adapted to allow liquid to exit the reaction vessel;
a second conduit in fluid communication with the reaction vessel, the second conduit is adapted to return the exited liquid to the reaction vessel; and
an oxygen entrainment section between the first and second conduits adapted to allow contact between the liquid from the first conduit and oxygen prior to the liquid returning to the reaction vessel.

8. The apparatus of claim 7, wherein the oxygen is contained in an oxygen-containing fluid selected from oxygen, air and oxygen-enriched air.

9. The apparatus of claim 7, wherein the circulation loop comprises a liquid propelling means for driving the liquid in the reaction vessel to flow through the loop in a desired direction.

10. The apparatus of claim 9 wherein the liquid propelling means is a pump.

11. The apparatus of claim 7, wherein the location where liquid exits the reaction vessel into the first conduit is positioned higher than the location where liquid enters the reaction vessel from the second conduit.

12. The apparatus of claim 7, wherein at least a portion of the circulation loop is positioned lower than the reaction vessel.

13. A method for constructing an apparatus comprising:
converting a carbon source into a hydrocarbon or hydrocarbon precursor via a microorganism-catalysed aerobic reaction, the method comprising fitting a reaction vessel having a capacity of about 2000 litres or greater with an aeration system for supplying oxygen to the vessel.

14. The method of claim 1 further comprising the step of producing a biofuel component using the hydrocarbon or hydrocarbon precursor.

15. The method of claim 14 further comprising subjecting the hydrocarbon or hydrocarbon precursor to a downstream hydrotreatment.

16. The apparatus of claim 7 wherein the circulation loop further comprises a section having an essentially U-shape.

17. The apparatus of claim 7 further comprising a stirring element configured to cause movement in the liquid in the reaction vessel.

18. The apparatus of claim 7 further comprising a temperature controlling element.

19. The apparatus of claim 7 wherein liquid returning to the vessel reaction from the second conduit has a higher oxygen concentration than the liquid exiting the reaction vessel to the first conduit.

20. The apparatus of claim 19 wherein the higher oxygen concentration is caused at least by the depth at which a portion of the circulation loop is positioned below the reaction vessel.
